(19) United States
(12) Patent Application Publication
(10) Pub. No.: US 2011/0118218 A1
(43) Pub. Date: May 19, 2011

(54) TREATING HYPERTENSION WITH 25-HYDROXYVITAMIN D3

(76) Inventors: Neil Robert Buck, Leymen (FR); Wouter Claerhout, Singapore (SG); Bruno H. Leuenberger, Rheinfelden (CH); Elisabeth Stoecklin, Arlesheim (CH); Kai Urban, Bad Sackingen (DE); Swen Wolfram, Waldshut-Tiengen (DE)

(21) Appl. No.: 12/867,309

(22) PCT Filed: Feb. 12, 2009
(86) PCT No.: PCT/EP09/51635

§ 371 Date: Jan. 31, 2011

Related U.S. Application Data
(60) Provisional application No. 61/028,510, filed on Feb. 13, 2008.
(60) Provisional application No. 61/031,671, filed on Feb. 26, 2008.
(60) Provisional application No. 61/036,926, filed on Mar. 14, 2008.
(60) Provisional application No. 61/036,928, filed on Mar. 15, 2008.

(51) Int. Cl. A61K 31/593; C07C 401/00; A61P 9/12

(52) U.S. Cl. 514/168; 514/167; 552/653

(57) ABSTRACT

We disclose the use of optionally in combination with vitamin D3 (cholecalciferol), 25-hydroxyvitamin D3 (cal-cifediol), to treat hypertension. Forms and dosages of a pharmaceutical composition, as well as processes for manufacturing medicaments, are also disclosed.

### FIELD OF THE INVENTION

The present invention relates to use of 25-hydroxyvitamin D3 (calcifediol) or a combination of 25-hydroxyvitamin D3 and Vitamin D to treat hypertension or to maintain healthy blood pressure levels.

### BACKGROUND OF THE INVENTION

Vitamin D (e.g., ergocalciferol and cholecalciferol) is a group of fat-soluble compounds defined by their biological activity. A deficiency of vitamin D causes rickets in children and osteomalacia in adults. But toxicity can occur after chronic intake of more than 100 times the recommended daily allowance (i.e., 5-15 μg or 200-600 IU vitamin D) for several months. For vitamin D, “The threshold for toxicity is 500 to 600 mcg/kg body weight per day. In general, adults should not consume more than three times the RDA for extended period of time” (Garrison & Somer, The Nutrition Desk Reference, Third Ed., McGraw-Hill, pg. 82, 1997). Hypercalcemia may occur at a blood concentration of 25-hydroxyvitamin D3 greater than 375 nmol/L. More recently, a safe upper level of vitamin D3 was identified to be at least 250 μg/day (Hathcock et al. Am. J Clin. Nutr. 85:6-18, 2007). Ingestion of such as a dietary supplement has been shown to result in a blood concentration of about 200 nmol/L 25-hydroxyvitamin D3.

Vitamin D is a prohormone. Vitamin D3 (cholecalciferol) is hydroxylated in the liver to produce 25-hydroxyvitamin D3 (calcifediol, 25-OH vitamin D3, 25-OH D3), which then undergoes another hydroxylation in the kidney and other tissues to produce 1,25-dihydroxyvitamin D3 (calcitriol), the active hormone form of vitamin D. Calcitriol is released into the blood, binds to vitamin D binding protein (DBP), and is transported to target tissues. Binding between calcitriol and vitamin D receptor allows the complex to act as a transcription factor in the cell's nucleus. Vitamin D regulates calcium and phosphorus concentrations in the blood; it promotes bone formation and mineralization. But at very high levels, vitamin D may promote resorption of bone. It may also modulate function of the cardiovascular, immune, and muscular systems. Epidemiological studies find associations between vitamin D intake and its effect on blood pressure or glucose metabolism. The activity of vitamin D is under negative feedback control by parathyroid hormone. Both Vitamin D and 25-OH D3 have been administered as pharmaceuticals in the past.

Vitamin D, is of course widely available; 25-OH D3 was previously sold in the USA by Organon USA under the name “CALDEROL”, but is currently on the FDA's list of discontinued drugs. It was a gelatine capsule containing corn oil and 25-OH D3.

A liquid for of 25-OH D3 is currently sold in Spain by FAES Farma under the name “HIDROFEROL” in an oil solution.

The combination of Vitamin D3 and 25-OH D3 has been used in animal feed. 25-OH D3 for use in feed is commercially available from DSM under the name “ROVIMIX HY-D”.

Tritsch et al. (US 2003/0170324) disclose a feed premix composition of at least 25-OH D3 in an amount between 5% and 50% (wt/wt) dissolved in oil, an agent encapsulating droplets of 25(OH)D3 and oil, and a nutritional additive (e.g., vitamin D3). The premix may be added to poultry, swine, canine, or feline food. This composition stabilized 25(OH)D3 against oxidation.

Simoes-Nunes et al. (US 2005/0064018) disclose adding a combination of 25(OH)vitamin D3 and vitamin D3 to animal feed. In particular, about 10 μg/kg to about 100 μg/kg of 25(OH)vitamin D3 and about 200 IU/kg to about 4,000 IU/kg of vitamin D3 are added to swine feed. This addition improves the pig's bone strength.

Stark et al. (U.S. Pat. No. 5,695,794) disclose adding a combination of 25-OH D3 and vitamin D3 to poultry feed to ameliorate the effects of tibial dyschondroplasia.

Chung et al, WO 2007/059960 discloses that sows fed a diet containing both Vitamin D3 and 25-OH D3 had improved general health status, body frame, litter size and health, and other production parameters. Also a 25-OH D3 human food supplement is disclosed, but its dosage range, 5-15 micrograms per kg body weight, is very high.

Li et al. 2002 J. Clin. Invest. 110:229-238 disclose renin suppression by injecting mice with 1,25(OH)₂vitamin D3. They suggest that vitamin D analogs could help prevent or ameliorate hypertension.

To our knowledge the prior art does not teach or suggest use of 25-hydroxy vitamin D3 alone or in combination with Vitamin D3 as a medicament for humans to treat hypertension.

### SUMMARY OF THE INVENTION

It has been found in accordance with this invention, that 25-OH D3, administered either alone in combination with Vitamin D3 can be used as a medicament or nutraceutical, food supplement or food to maintain healthy blood pressure, to prevent high blood pressure, and to lower levels of high blood pressure.

In one embodiment of this invention, a method of administering, 25-OH D3, or both 25-OH D3 and vitamin D to a human is provided. As a result, blood pressure may be reduced to or maintained at a normal level (e.g., systolic blood pressure less than 120 mm Hg and/or diastolic blood pressure less than 80 mm Hg). Dosages may be administered once per day, once per week, or once per month.

In another aspect, a pharmaceutical composition suitable for human use is provided which comprises vitamin D3, 25-hydroxyvitamin D3, and a pharmaceutically acceptable carrier in amounts to normalize blood pressure in a human.

In yet another aspect of this invention, 25-OH D3 or the combination of 25-OH and Vitamin D is administered along with conventional hypertension therapy in order to provide a sustained relief from high blood pressure.

As used throughout the specification and claims, the following definitions apply:

“Vitamin D” means either Vitamin D3 (cholecalciferol) and/or Vitamin D2 (ergocaciferol). Humans are unable to make Vitamin D2 (ergocalciferol), but are able to use it as a source of Vitamin D. Vitamin D2 can be synthesized by various plants and is often used in Vitamin D in supplements as an equivalent to Vitamin D.

“Vitamin D metabolite” means any metabolite of Vitamin D other than 25-hydroxy vitamin D3.

“25-OH D3” refers specifically to 25-hydroxyvitamin D3

“25-OH D” refers to the 25-hydroxylated metabolite of either Vitamin D2 or Vitamin D3 which is the major circulating form found in plasma.

“Prevent” is meant to include amelioration of the disease, lessening of the severity of the symptoms, early intervention, and lengthening the duration of onset of the disease, and not intended to be limited to a situation where the patient is no longer able to contract the disease nor experience any symptoms.

Blood pressure may be measured with a manual or automatic manometer. The “normal” (arterial) blood pressure has been progressively lowered to 115/75 mm Hg by medical authorities.

### DESCRIPTION OF SPECIFIC EMBODIMENTS OF THE INVENTION

Vitamin D3 and 25-hydroxyvitamin D3 may be obtained from any source, and a composition thereof may be prepared using convenient technology. In general, crystals of vitamin D3, 25-hydroxyvitamin D3, or both (separately or together) are dissolved in an oil with heating and agitation. Preferably, the oil is transferred into a vessel and heated. Thereafter, vitamin D3, 25-hydroxyvitamin D3, or both are added to the vessel, while maintaining the temperature of the oil or increasing it over time. The composition is agitated to dissolve the crystals of vitamin D3, 25-hydroxyvitamin D3, or both. Prior to addition to the oil, the crystals may be reduced in size by milling and/or sieving, to enhance dissolving. The composition may be agitated by stirring, vessel rotation, mixing, homogenization, recirculation, or ultrasonication. Preferably, the oil may be heated in the vessel to a temperature from about 80° C. to about 85° C., sized crystals are introduced into the vessel, and the contents are stirred to dissolve the crystals into the oil.

The “oil” may be any edible oil, lipid, or fat: e.g., babassu oil, coconut oil, cohune oil, murumyru tallow, palm kernel oil, or tucum oil. The oil may be natural, synthetic, semisynthetic, or any combination thereof. Natural oil may be derived from any source (e.g., animal, plant, fungal, marine); synthetic or semisynthetic oil may be produced by convenient technology. Preferably, the oil is a mixture of plant medium chain triglycerides, mainly caprylic and capric acids. The composition may optionally contain one or more other suitable ingredients such as, for example, antioxidants, preservatives, dissolution agents, surfactants, pH adjusting agents or buffers, humectants, and any combination thereof. The foregoing are examples of pharmaceutically acceptable carriers.

Suitable antioxidants are those approved for human pharmaceutical use, and include tocopherol, mixed tocopherols, tocopherols from natural or synthetic sources, butylated hydroxy toluene (BHT), butylated hydroxy anisole (BHA), natural antioxidants like rosemary extract, propyl galate, and any others used in the manufacture of pharmaceuticals for humans. Preferably, the antioxidant is tocopherol. Suitable preservatives include methyl paraben, propyl paraben, potassium sorbate, sodium benzoate, benzoic acid, and any combination thereof. Suitable dissolution agents include inorganic or organic solvents: e.g., alcohols, chlorinated hydrocarbons, and any combination thereof. Suitable surfactants may be anionic, cationic, or nonionic: e.g., ascorbyl palmitate, polysorbates, polyethylene glycols, and any combination thereof. Suitable pH adjusting agents or buffers include citric acid-sodium citrate, phosphoric acid-sodium phosphate, acetic acid-sodium acetate, and any combination thereof. Suitable humectants include glycerol, sorbitol, polyethylene glycol, propylene glycol, and any combination thereof.

Once formed, the oil composition may be incorporated in various other useful compositions, some of which are discussed below. For example, emulsions may be formed, which may be optionally encapsulated or spray dried. A variety of emulsions may be prepared by combining the nonaqueous compositions described above with an aqueous composition. The emulsion may be of any type. Suitable emulsions include oil-in-water emulsions, water-in-oil emulsions, anhydrous emulsions, solid emulsions, and microemulsions. The emulsions may be prepared by any convenient technology. The emulsion contains an aqueous composition and a nonaqueous (e.g., oil) composition, wherein the latter comprises vitamin D3, 25-hydroxyvitamin D3, or both (separately or together) dissolved in an oil in an amount of between about 3% and about 50% by weight based on the total weight of the oil composition. As used herein, “aqueous composition” and “aqueous phase” are used interchangeably. Generally, the emulsion may contain from about 20% to about 95% of an aqueous composition, and from about 5% to about 80% of a nonaqueous composition. Preferably, however, the emulsion contains from about 85% to about 95% (vol/vol) of an aqueous composition, and from about 5% to about 15% (vol/vol) of a nonaqueous composition. Conveniently, the nonaqueous composition may be dispersed as droplets in the aqueous composition. For example, the droplets may have a mean diameter of less than about 500 nm in the aqueous composition. Conveniently, the droplets have a mean diameter of between about 100 nm and about 200 nm.

In a particularly advantageous embodiment, the emulsion contains an encapsulating agent, which facilitates encapsulating the oil composition upon further processing of the emulsion (e.g., by spray drying). The encapsulating agent may be any edible substance capable of encapsulating the oil composition. Preferably, the encapsulation agent is predominantly a colloidal material. Such materials include starches, proteins from animal sources (including gelatins), proteins from plant sources, casein, pectin, alginate, agar, maltodextrins, lignin sulfonates, cellulose derivatives, sugars, saccharides, sorbitols, gums, and any combination thereof.

Suitable starches include: plant starches (e.g., CAPSUL® or HI-CAP® from National Starch & Chemical Corp., New York, N.Y.), other modified food starches, and any combination thereof. Preferably, the starch is CAPSUL® modified plant starch. Suitable proteins from animal sources include: gelatins (e.g., bovine gelatins, porcine gelatins (Type A or B) with different Bloom numbers, fish gelatins), skim milk protein, caseinate, and any combination thereof. Preferably, the animal protein is a gelatin. Suitable proteins from plant sources include: potato protein (e.g., ALBUREX® from Roquette Preres Societe Anonyme, Lestrem, France), pea protein, soy protein, and any combination thereof. Preferably, the plant protein is ALBUREX® potato protein. Suitable maltodextrins with a different dextrose equivalent include: maltodextrin 5, maltodextrin 10, maltodextrin 15, maltodextrin 20, maltodextrin 25, and any combination thereof. Preferably, the maltodextrin is maltodextrin 15. Suitable cellulose derivatives include: ethyl cellulose, methylethyl cellulose, hydroxypropyl cellulose, hydroxypropylmethyl cellulose, carboxymethylcellulose, and any combination thereof. Suitable saccharides include lactose, sucrose, or any combination thereof. Preferably, the saccharide is sucrose. Suitable gums include: acacia, locust bean, carragean, and any combination thereof. Preferably, the gum is gum acacia.

When the emulsion contains an encapsulating agent, the encapsulating agent may be dispersed in water by any convenient technology to form an aqueous phase. The aqueous phase may be a solution or a mixture depending on the properties of the components selected. The selected components may be dispersed by any convenient technology including: homogenizing, mixing, emulsifying, recirculating, static mixing, ultrasonication, stirring, heating, or any combination thereof. The viscosity of the resulting aqueous phase may then be adjusted, as desired, by the addition of water. The aqueous composition of the emulsion may optionally contain any other suitable material including but not limited to, those discussed above in reference to the nonaqueous composition. Preferably, the aqueous composition may include, an encapsulating agent, a film-forming agent, a plasticizer, a preservative, an antioxidant, or any combination thereof. Suitable preservatives include methyl paraben, propyl paraben, sorbic acid, potassium sorbate, sodium benzoate, and any combination thereof. Suitable antioxidants include sodium ascorbate, ascorbic acid, citric acid, and any combination thereof.

Preferably, the aqueous phase contains a modified food starch, such as octenyl succinyl starch (CAPSUL®), maltodextrin, and sodium ascorbate. Another preferred aqueous phase contains potato protein (ALBUREX®), maltodextrin 20, and sodium ascorbate. The selected components may be dissolved in water by any convenient technology, preferably stirring. The mixture is preferably homogenized until it is uniform and lump free. Preferably, the homogenization is carried out at a temperature between about 50° C. and about 75° C. The final viscosity of the resulting aqueous phase may then be adjusted to the desired viscosity, preferably about 250 cp to about 450 cp, more preferably about 300 cp to about 400 cp, even more preferably about 385 cp.

The emulsion may be formed by emulsifying the nonaqueous composition and the aqueous phase by any means, including homogenization, rotor-stator shear, high pressure shear and cavitation, high speed “cowles” or shear agitation, and any combination thereof. The volume and viscosity of the emulsion may preferably be adjusted by the addition of water after emulsification. Preferably, the nonaqueous and aqueous compositions are emulsified by homogenization. Preferably, the emulsion should not contain any mineral, transition metal, or peroxide.

As noted above, the emulsion may be incorporated or employed in producing other useful compositions, especially encapsulated oils, e.g., spray-dried powders. Generally, the encapsulated oil comprises an oil composition and an encapsulation agent encapsulating the oil composition, wherein the oil composition contains vitamin D3, 25-hydroxyvitamin D3, or both dissolved in the oil in an amount between about 3% and about 50% by weight based on the total weight of the oil composition. The encapsulated oil may be produced by any convenient technology: e.g., drying an emulsion described above by any conventional technology, including spray drying, freeze drying, fluid bed drying, tray drying, adsorbtion, and any combination thereof. Preferably, the encapsulated oil is produced by spray drying an emulsion having an aqueous phase above containing an encapsulation agent; spray drying parameters are dictated by the physical characteristics desired in the final encapsulated oil. Such physical parameters include particle size, powder shape and flow, and water content. Preferably, the oil is in an amount less than about 30%, less than about 20%, less than about 10%, or less than about 5% by weight based on the total weight of the encapsulated oil. The encapsulated oil should have good flowability and the vitamin D3 and/or 25-hydroxyvitamin D3 should be distributed homogeneously throughout the composition. Conveniently, the encapsulated oil is a powder. Any other suitable additive may be added to the encapsulated oil. One such additive may be a flow agent such as silicon dioxide, to increase the flowability of the encapsulated oil.

The composition may be provided in the form of a tablet, capsule (e.g., hard or soft), or injection (e.g., oil or emulsion). They may be packaged in a single daily dosage.

### Dosages

Daily. A composition according to this invention where the two active ingredients are to be administered separately or alone, contains Vitamin D or 25-OH D3 in an amount from about 1 μg to about 50 μg, preferably about 5 μg and 25 μg. Alternatively, a single daily dosage having both Vitamin D and 25-OH D3 contains each active ingredient in an amount from about 1 μg to about 50 μg, preferably about 5 μg and 25 μg.

The dosage ratio of Vitamin D to 25-OH D3 may be from about 50:1 to about 1:50, more preferably from about 25:1 to about 1:25, and even more preferably from about 6:1 to about 1:6.

Multiple, separate dosages may be packaged in a single kit (or container). For example, the kit may be comprised of thirty separate daily dosages of both actives separately (i.e. 60 separate dosages), or combined (i.e. 30 dosages containing both active ingredients). Instructions for administering the dosages to a human may be included in the kit.

Weekly. A single weekly dosage contains Vitamin D or 25-OH D3 in an amount from about 7 μg to about 350 μg, and preferably from about 35 to 175 μg. Alternatively, a single weekly dosage may contain both Vitamin D and 25-OH D3 each in an amount from about 7 μg to about 350 μg, and preferably from about 35 to 175 μg . The dosage ratio of Vitamin D to 25-OH D3 may be from about 50:1 to about 1:50, more preferably from about 25:1 to about 1:25, and even more preferably from about 6:1 to about 1:6.

Monthly. A single monthly dosage contains Vitamin D or 25-OH D3 in an amount from 30 μg to about 1500 μg, preferably about 75 μg to about 500 μg. Alternatively, a single monthly dosage may contain both Vitamin D and 25-OH D3 each in an amount from 30 μg to about 1500 μg, preferably about 75 μg to about 500 μg. A kit may be comprised of one, two, three, four, five, six, seven, eight, nine, ten, eleven, or twelve weekly or monthly dosages.

Dosage ratios of Vitamin D to 25-OH D3 should range between 50:1 to about 1:50, more preferably from about 25:1 to about 1:25, and even more preferably from about 6:1 to about 1:6.

Another aspect of this invention is the concommittant administration of 25-OH D3, alone or in combination with vitamin D3 along with a conventional pharmaceutical recognized to control blood pressure. A lower dose of the pharmaceutical is required when it is administered with 25-OH D3, than when it is administered alone, and as a result, the patient is less at risk for possible side effects of the pharmaceutical, or less at risk from drug-drug interactions.

Examples of conventional pharmaceuticals used to control or decrease hypertension include:

Diuretics such as: Chlorthalidone, Hydrochlorothiazide, Indapamide, Metolazone

Loop diuretics such as: Bumetanide, Ethacrynic acid, Furosemide, Torsemide

Potassium-sparing agents such as: Amiloride hydrochloride, Spironolactone, Triamterene

Adrenergic inhibitors:
Peripheral agents such as: Reserpine
Central alpha-agonists such as: Clonidine hydrochloride, Guanabenz acetate, Guanfacine hydrochloride, Methyldopa
Alpha-blockers such as: Doxazosin mesylate, Prazosin hydrochloride, Terazosin hydrochloride
Beta-blockers such as: Acebutolol, Atenolol, Betaxolol, Bisoprolol fumarate,Carteolol hydrochloride, Metoprolol tartrate, Metoprolol succinate, Nadolol, Penbutolol sulfate, Pindolol, Propranolol hydrochloride, Timolol maleate
Combined alpha- and beta-blockers such as: Carvedilol, Labetalol hydrochloride

Direct vasodilators such as: Hydralazine hydrochloride, Minoxidil

Calcium antagonists
Nondihydropyridines such as: Diltiazem hydrochloride,Verapamil hydrochloride
Dihydropyridines such as Amlodipine besylate, Felodipine, Isradipine Nicardipine, Nifedipine, Nisoldipine

ACE inhibitors such as: Benazepril hydrochloride, Captopril, Enalapril maleate, Fosinopril sodium, Lisinopril, Moexipril, Quinapril hydrochloride, Ramipril Trandolapril

Angiotensin II receptor blockers such as: Losartan potassium, Valsartan, Irbesartan

Various combinations of these or similiar anithypertensive agents agents are also available.

Contributing factors to hypertension include low calcium, abnormal regulation of the renin-angiotensin system, and insulin resistance. Hypertension increases the risk for atherosclerosis, heart attack, heart failure, nephropathy, retinopathy, and stroke.

One aspect of this invention is the use of 25-OH D3 in the manufacture of a medicament, food, or nutraceutical useful in lowering high blood pressure, preventing high blood pressure, and/or maintaining normal blood pressure.

### EXAMPLES

### Example 1

### Human Clinical Trial

### Materials and Methods

Spray-dried formulation of 25-hydroxyvitamin D3 was provided as a powder. In summary, 25-hydroxyvitamin D3 and DL-α-tocopherol were dissolved in an oil of medium chain triglycerides, then emulsified into an aqueous solution of modified starch, sucrose, and sodium ascorbate. The emulsion was atomized in a spray dryer in the presence of silicon dioxide. The resulting powder was collected when water content (LDO) was less than 4% and sieved through 400 μm. It was packed and sealed in alu-bags, then stored in a dry area below 15° C. and used within 12 months of its manufacture.

Three separate lots were manufactured. In detail, a matrix was produced by mixing for 120 min in a FRYMIX processing unit with an anchor stirrer at 70° C. under vacuum and consisting of:

17.300 kg water (WBI)

13.46 kg modified food starch (CAPSUL HS)

3.270 kg sucrose

0.730 kg sodium ascorbate

An oil phase was prepared by mixing for 35 min in a double-walled vessel with propeller stirrer at 82° C. and consisting of:

0.550 kg BERGABEST MCT oil 60/40

0.049 kg calcifediol (HY-D USP)

0.183 kg DL-α-tocopherol

The oil phase was transferred to the matrix in the FRYMIX processing unit and was pre-emulsified with its internal colloid mill (60 min, 70° C.). The pre-emulsion was circulated through a high-pressure homogenizer (20 minr) The emulsion with a viscosity of 60 mPa·s to 90 mPa·s at 70° C. was transferred over the high pressure pump to the spray nozzle. As fluidizing agent, silicon dioxide (SIPERNAT 320 DS) was fed into the tower. The spraying and drying parameters are listed below.

| Parameter | Spraying | | Drying | |
|---|---|---|---|---|
| Inlet air position | top of tower | | top of tower | |
| Inlet air feed | 1500 | m³/h | 1400 | m³/h |
| Inlet air temperature | 170 | C. | heater switch off | |
| IFB inlet air feed | 500 | m³/h | 500 | m³/h |
| IFB inlet air temperature | 65° | C. | 50° | C. |

| exhaust air position | bottom of the tower | | bottom of the tower |
|---|---|---|---|
| fine powder recycling | to IFB | | to IFB |
| emulsion feed rate | 50 | kg/h | emulsion feed stopped |
| SiO₂ feed position | top of tower | | SiO₂ feed stopped |
| SiO₂ acid feed rate | 100 | g/h | SiO₂ feed stopped |

For each of the three lots of 25-hydroxyvitamin D3, an average of 8.4 kg of spray-dried powder with about 0.25% content of 25-hydroxyvitamin D3 was obtained. The other components of the formulation are: 73.2% modified food starch, 17.6% sucrose, 4.0% sodium ascorbate, 3.0% medium chain triglycerides, 1.0% silicon dioxide, and 1.0% DL-α-tocopherol.

Spray-dried formulation of vitamin D3 was provided as a powder. In summary, vitamin D3 and DL-α-tocopherol were dissolved in an oil of medium chain triglycerides, then emulsified into an aqueous solution of modified starch, sucrose, and sodium ascorbate. The emulsion was atomized in a spray dryer in the presence of silicon dioxide. The resulting powder was collected when water content (LOD) was less than 4% and sieved to remove big lumps. It was stored in a dry area below 15° C. and used within 12 months of its manufacture.

### Subjects

Healthy, postmenopausal women (50 to 70 years of age) were recruited using informed consent and screened using the following criteria: serum 25-hydroxy vitamin D3 between 20 nmol/L and 50 nmol/L, body mass index between 18 kg/m² and 27 kg/m², blood pressure less than 146/95 mm Hg, serum calcium less than 2.6 nmol/L, fasting glucose less than 100 mg/dl, no high-intensity exercise more than three times per week, no treatment for hypertension, no use of high-dose vitamin D or calcium supplement or drug affecting bone metabolism (e.g., biphosphonate, calcitonin, estrogen receptor modulator, hormone replacement therapy, parathyroid hormone), and not visiting a “sunny” location during the study.

Subjects are randomly assigned to one of seven treatment groups (i.e., daily, weekly, bolus as single dose, and bolus as combination dose). Each group includes five subjects. They are followed for four months in Ziirich, Switzerland during the winter.

### Clinical Study

The objective was studying and comparing the pharmacokinetic characteristics of vitamin D3 and 25-hydroxyvitamin D3 administered to humans. Equimolar quantities of both substances were investigated. The regimen is based on 20 μg/day (or its equivalent on a weekly basis) of 25-hydroxyvitamin D3. As the maximum pre-existing baseline concentration of 25-hydroxyvitamin D3 will be 50 nmol/L, it is not anticipated that subjects will approach the range where disturbance in Ca²⁺ homeostasis has been observed. For comparative purposes, it was necessary to administer equimolar quantities of either vitamin D3 or 25-hydroxyvitamin D3. In respect to administration of vitamin D3, the dose is considered to be sufficient to overcome background variability and provide and efficacious dose to the participants.

Daily: 120 administrations

| | | |
|---|---|---|
| 1. | 25-Hydroxyvitamin D3 | 20 μg |
| 2. | Vitamin D3 | 20 μg (800 IU) |

Weekly: 16 administrations

| | | |
|---|---|---|
| 3. | 25-Hydroxyvitamin D3 | 140 μg |
| 4. | Vitamin D3 | 140 μg (5600 IU) |

Bolus: single administration

| | | |
|---|---|---|
| 5. | 25-Hydroxyvitamin D3 | 140 μg |
| 6. | Vitamin D3 | 140 μg (5600 IU) |

Bolus: combo administration

| | | |
|---|---|---|
| 7. | D3 and 25(OH)D3 | 140 μg (5600 IU) + 140 μg |

Hard gel capsules, which are packaged in bottles, contain either 20 μg or 140 μg of either spray-dried vitamin D3 or 25-hydroxyvitamin D3 per capsule. Each dosage was consumed orally at breakfast. The duration of the study was four months for the “Daily” and “Weekly” groups. Subjects enrolled in the “Bolus” group consumed orally a single dosage at the second study visit.

Plasma concentrations of 25-hydroxyvitamin D3 (e.g., peak and steady state) were determined by obtaining samples from the subjects at various times after the dosage is ingested. For screening purposes and to establish baseline values, a blood sample was obtained prior to enrollment into the study and the clinical laboratory measured vitamin D3, 25-hydroxyvitamin D3, calcium, creatinine, albumin, and fasting glucose in the serum. On

Monday of Week 1 of the study, pharmacokinetics of serum vitamin D3, 25-hydroxyvitamin D3, and 1,25-dihydroxyvitamin D3; serum markers (i.e., vitamin D3, 25-hydroxyvitamin D3, calcium, creatinine, albumin, PTH, GOT, GPT, ALP, triglycerides, HDL, LDL, total cholesterol, bALP, and fasting glucose); and urine markers (i.e., calcium, creatinine, and DPD) are assessed over 24 hours. Daily samples for the remaining days of Week 1 and Monday of Week 2 are taken to assess serum vitamin D3 and 25-hydroxyvitamin D3, serum markers (i.e., calcium, creatinine, albumin), and urine markers (i.e., calcium, creatinine). The assessments continue on Monday of Weeks 3, 5, 7, 9, 11, 13 and 15. On Monday of Week 16, samples are taken to assess pharmacokinetics of serum vitamin D3, 25-hydroxyvitamin D3, and 1,25-dihydroxyvitamin D3; serum markers (i.e., vitamin D3, 25-hydroxyvitamin D3, calcium, creatinine, albumin, PTH, GOT, GPT, ALP, triglycerides, HDL, LDL, total cholesterol, bALP, and fasting glucose); and urine markers (i.e., calcium, creatinine, and DPD).

For blood pressure assessments a standard protocol was used (OMRON professional apparatus) to measure diastolic and systolic blood pressure in Week 1 on visit 2 (baseline) and the remaining 4 days of Week 1. The assessments continued at each visit of Weeks 2, 3, 5, 7, 9, 11, 13 and 15.

Table 1 shows blood pressure after daily and weekly treatment with 25-OH D3 (20 μg per day; 140 μg per week, respectively) and daily and weekly treatment with Vitamin D3 (20 μg per day; 140 μg per week, respectively). Treatment duration was 4 months. Values are GLM (general linear model) least square means given as mmHg after repeated measurement analysis over 13 visits adjusted for baseline blood pressure and time for 25-OH D3 versus Vitamin D3.

| | Blood pressure (in mmHg) | |
|---|---|---|
| | Daily/weekly | Daily/weekly |
| | Vitamin D3 | 25-OH D₃ |
| Systolic blood pressure | 117.2 | 111.1 |
| Diastolic blood pressure | 70.3 | 68.9 |

Table 2: Change in blood pressure after daily and weekly treatment with 25-OH D3 (20 μg per day; 140 μg per week, respectively) compared to daily and weekly treatment with Vitamin D3 (20 μg per day; 140 μg per week, respectively). Treatment duration was 4 months. Values are given as mmHg reduction of blood pressure after repeated measurement analysis over 13 visits adjusted for baseline blood pressure and time for 25-OH D3 versus Vitamin D3.

| | Blood pressure (Change vs. |
|---|---|
| | Daily/weekly Vitamin D₃ in mmHg) |
| | Daily/weekly 25-OHD3 |
| Systolic blood pressure | −6.1 |
| Diastolic blood pressure | −1.4 |

These data demonstrate that daily or weekly treatment with 25-OH D3 surprisingly results is much stronger reductions of blood pressure compared to consumption of identical dosages of Vitamin D3. After treatment with 25-OH D3 subjects displayed more pronounced reductions in blood pressure compared to before treatment and compared to treatment with Vitamin D3. The reduction of systolic and diastolic blood pressure in subjects treated with 25-OH D3 versus Vitamin D3 was 6.1 mmHg and 1.4 mmHg, respectively, an effect size that is clinically relevant and represents a significant benefit for subjects in all age groups.

The magnitude of the reduction observed in the present study is even more important due to the fact that subjects in the present study were only mildly hypertensive and no subjects with a blood pressure above 146/95 mmHg were included in the study. In mildly hypertensive subjects, the achievable reduction is generally small with currently marketed anti-hypertensive drugs. Therefore, treatment with 25(OH)D3 surprisingly resulted in a very effective reduction of blood pressure whereas no effect was observed for Vitamin D3.

The invention described and claimed herein is not to be limited in scope by the specific embodiments herein enclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In case of conflict, the present disclosure including definitions will control.

1. A method of treating a human comprising administering 25-hydroxyvitamin D3 (25-0H D3) or a combination of 25-OH D3 and vitamin D3, to a human in an amount sufficient to lower or maintain systolic blood pressure to less than 120 mm Hg and/or diastolic blood pressure to less than 80 mm Hg.

2. The method according to claim 1, wherein vitamin D3 and 25-hydroxyvitamin D3 are administered separately to the human.

3. The method according to claim 1, wherein vitamin D3 and 25-hydroxyvitamin D3 are administered together to the human.

4. The method according to claim 1, wherein the human is treated once daily, weekly, or monthly.

5. Use of 25-OH D3 and optionally Vitamin D3 to lower or maintain systolic blood pressure to less than 80 mm Hg.

6. Use of 25-OH D3 in the manufacture of a medicament, nutraceutical, food supplement or food which can lower or maintain systolic blood pressure to less than 80 mm Hg.

7. A composition comprising (i) vitamin D3 and 25-hydroxyvitamin D3 in amounts sufficient to lower or maintain systolic blood pressure to less than 120 mm Hg and/or diastolic blood pressure to less than 80 mm Hg and (ii) a pharmaceutically-acceptable carrier.
